(19) United States
(12) Patent Application Publication
(10) Pub. No.: US 2015/0005179 A1
(43) Pub. Date: Jan. 1, 2015

(54) HLA PRIMERS FOR PERSONAL COMPATIBILITY

(71) Applicant: Instant Chemistry Inc., Mississauga (CA)

(72) Inventor: Sara Seabrooke, Mississauga (CA)

(21) Appl. No.: 13/929,024

(22) Filed: Jun. 27, 2013

(51) Int. Cl. C12Q 1/68

(52) U.S. Cl. CPC C12Q 1/6853
USPC 506/7; 506/16

(57) ABSTRACT

Processing personal compatibility matches includes a computer system receiving input of a first allele group determined to be present in a first person and a second allele group determined to be present in a second person. The first allele group and the second allele group are members of a set of allele groups for a human leukocyte antigen (HLA) gene. Each group of the set of allele groups is predefined to contain related alleles. By comparing the first allele group to the second allele group, a similarity of the first allele group to the second allele group may be determined. An indication of personal compatibility of the first person and the second person is inversely related to the determined similarity. The computer system can output the indication of personal compatibility to, for example, a matchmaker or a third-parting service.

### FIELD

This disclosure relates to obtaining and processing genetic information.

### REFERENCE TO SEQUENCE LISTING

A sequence listing in an ASCII text file (IC2-HLA_ST25.txt) accompanies this specification, as provided for by the EFS Legal Framework Notice of Apr. 6, 2011. The file was created on Jun. 27, 2013 and contains 21,747 bytes. The entire content of the sequence listing file is hereby incorporated by reference.

### BACKGROUND

Personal compatibility is important to long-term and satisfying relationships. There are many known techniques for increasing the likelihood that two people will find themselves to be compatible, and some of these techniques analyze genes. However, known techniques do not generally teach quantified genetic comparisons that can succinctly or accurately express the relatively complex differences between genes of the two individuals.

### SUMMARY

According to one aspect of the present disclosure, a primer array for use in analyzing personal compatibility based on human leukocyte antigen (HLA) genes includes at least one HLA-A primer set selected to target allele groups of an HLA-A gene of a DNA sample to undergo quantitative real-time polymerase chain reaction (qRT-PCR) processing, at least one HLA-B primer set selected to target allele groups of an HLA-B gene of the DNA sample to undergo qRT-PCR processing, and at least one HLA-DRB1 primer set selected to target allele groups of an HLA-DRB1 gene of the DNA sample to undergo qRT-PCR processing.

According to another aspect of the present disclosure, a method of processing a DNA sample extracted from a person to analyze personal compatibility includes extracting DNA from the DNA sample, performing polymerase chain reactions (PCRs) on extracted DNA to amplify HLA-A, HLA-B, and HLA-DRB1 genes, performing qRT-PCRs on products of the PCRs and on extracted DNA using at least one HLA-A primer set selected to target allele groups of an HLA-A gene, at least one HLA-B primer set selected to target allele groups of an HLA-B gene, and at least one HLA-DRB1 primer set selected to target allele groups of an HLA-DRB1 gene.

According to further aspects of the present disclosure, various primer sets and combinations thereof are disclosed.

### BRIEF DESCRIPTION OF THE DRAWINGS

The drawings illustrate, by way of example only, embodiments of the present disclosure.

FIG. 1 is a schematic diagram of a method of processing personal compatibility matches.

FIG. 2 is a schematic diagram of a data structure for personal compatibility matches using matchmakers.

FIG. 3 is a schematic diagram of a data structure for personal compatibility matches using third-party services.

FIG. 4 shows a block diagram of processing personal compatibility matches based on one or more HLA genes.

FIG. 5 shows a block diagram of processing personal compatibility matches based on predefined allele groups for HLA genes.

FIGS. 6a-6c are diagrams of condensed phylogenetic trees illustrating relationships among the predefined allele groups.

FIGS. 7a-7c are tables defining relationships among the predefined allele groups.

FIG. 8 shows an equation for determining a combined similarity result.

FIG. 9 is a table mapping similarity factor to personal compatibility.

FIG. 10 is a diagram of an output user interface for an interested person.

FIG. 11 is a diagram of an output user interface for a matchmaker.

FIG. 12 is a plan view of a primer plate.

FIG. 13 is a plan view of another primer plate.

FIG. 14 is chart of an experimental result.

### DETAILED DESCRIPTION

The present disclosure is directed to assessing personal (e.g., romantic or sexual) compatibility between two human beings based on a quantified comparison of the human leukocyte antigen (HLA) genes of the two individuals. The quantified comparison uses primers and calculation to arrive at a compatibility indication, which can succinctly and accurately express the relatively complex differences between HLA genes of the two individuals.

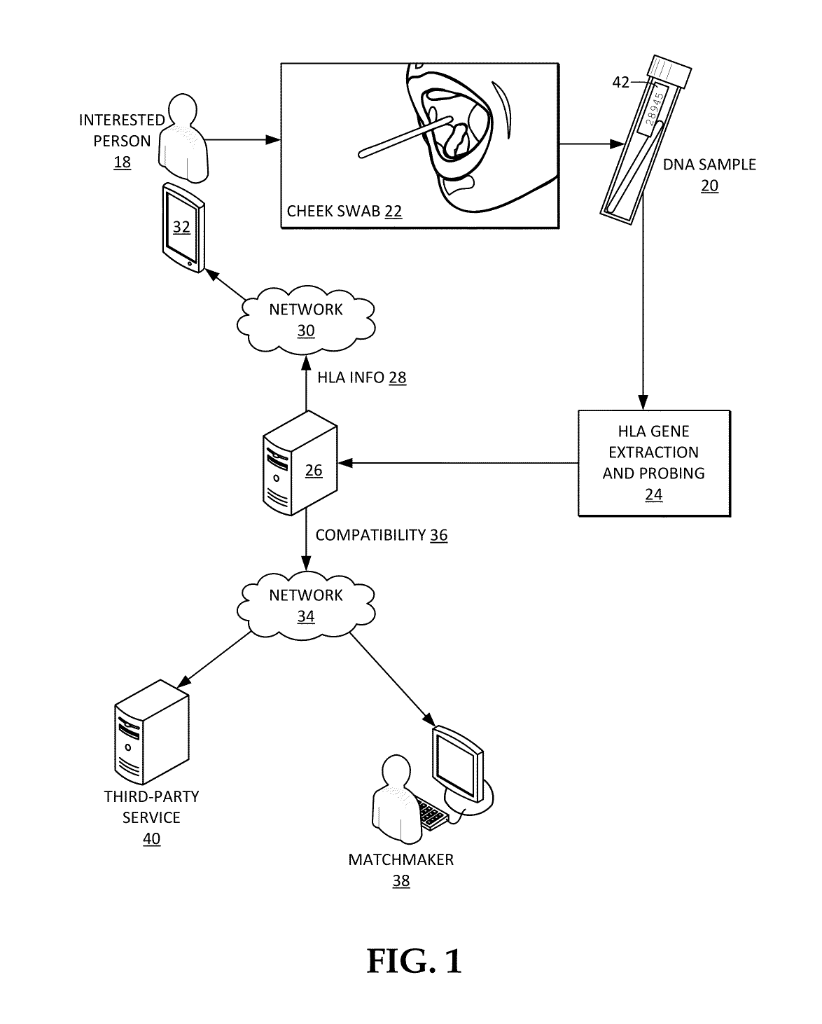

FIG. 1 illustrates a method of processing personal compatibility matches, such as romantic compatibility matches between two people.

An interested person 18 provides a DNA sample 20 by, for example, taking a cheek swab 22. A kit can be provided for this purpose, and such a kit can include a buccal swab and a sample container. Once the person has undergone the swab, the swab can be inserted into the sample container, which can then be mailed or otherwise delivered to a laboratory for processing. The kit can also a include instructions on how to perform the swab and a prepaid mailing envelope addressed to a suitable laboratory. More than one swab kit can be provided, so that more than one sample 20 can be taken, as a contingency against defective samples. When there is a matchmaker involved, swab kits may be provided to the matchmaker along with training, so that the matchmaker can request and instruct clients to perform a swab. Other techniques for obtaining DNA samples are also contemplated, such as hair samples, blood samples, skin samples, or samples of other DNA-bearing body tissue.

The laboratory performs DNA extraction and probing 24 for HLA genotyping of the interested individual, which is discussed in detail below. The HLA system contains genes related to immune system function in humans. Further, it has been discovered that HLA genes also play a role in human biological compatibility, in that the major histocompatibility complex (MHC) contributes to body scent in humans. Body scent and taste affect human sexual attraction and may help people, perhaps subconsciously, to find partners. Moreover, couples in long-term relationships tend to have MHC genes dissimilar to each other. Hence, the DNA extraction and probing 24 are performed on interested individuals to determine genetic information for HLA genes with the aim of determining HLA dissimilarity between two individuals so as to quantify the HLA aspect of attraction or compatibility between the two individuals.

HLA information resulting from the DNA extraction and probing 24 is provided to one or more server computers 26 connected to one or more computer networks 30, 34. The networks 30, 34 can be the same general network, such as the Internet. The server 26 and any connected remote computers or other electronic devices that receive output from the server 26 may be known as a computer system. The server 26 is configured to store and output the HLA information, and to process matches between selections of two individuals, one female and one male.

The server 26 can be configured to provide output of the interested person's HLA information 28 to the interested person 18. This can be performed by the server 26 outputting the interested person's HLA information 28 over the network 30 to an application at a computer or other electronic device 32 operated by the interested person 18. For example, the server 26 can output such information at a web page available to the interested person 18 via a log in or other credential verification.

The server 26 can be configured to provide output of an indication 34 of personal compatibility of two individuals based on the determined dissimilarity of their HLA genes. Output of the personal compatibility indication 36 can be provided over the network 34 in a form suitable for use by a computer or other electronic device operated by a third-party, such as a matchmaker 38. An example of output suitable for use by the matchmaker 38 includes a web page available to the matchmaker 38 via a log in or other credential verification. Alternatively or additionally, output of the personal compatibility indication 36 can be provided over the network 34 in a form suitable for use by a third-party service 40, such as a dating website or similar online personal service. An example of output suitable for use by the third-party service 40 includes a database connection to the server 26. Alternatively or additionally, output of the personal compatibility indication 36 can be provided to one or two of the interested persons in a romantic couple or potential couple (e.g., two people contemplating marriage).

The server 26 can be configured to restrict detailed HLA information to being output to the individual that provided the information, so as to protect privacy. This can be achieved by providing unique access codes 42 with the buccal swab kits, such as printed on a sticker or tag attached to the sample container. The unique access codes 42 are associated, at the server 26, to individual accounts assigned to interested persons 18. After a person's swab is processed, the HLA information is entered into the data store at the server 26 and associated with the unique access code 42, so that the person can then log in to their account to view their own HLA information. This can advantageously prevent the matchmaker 38, third-party service 40, or another entity from obtaining the private genetic information of the interested persons 18.

The server 26 can be configured to restrict personal compatibility indications 36 between two interested persons 18 to being output to one or more of the matchmaker 38 and the third-party service 40. This can be achieved by the server 26 storing and outputting personal compatibility indications 36 as indexed to non-private identities of two interested persons 18, such as two individual profile IDs, a match ID, or similar index. Such non-private identities are isolated from the unique access codes 42, so that knowledge of one or more non-private identities cannot be used to obtain a unique access code, and knowledge of a unique access code cannot be used to obtain other information. This can advantageously prevent an interested person 18 from obtaining information about potential matches outside of the context of the matchmaker 38 or third-party service 40.

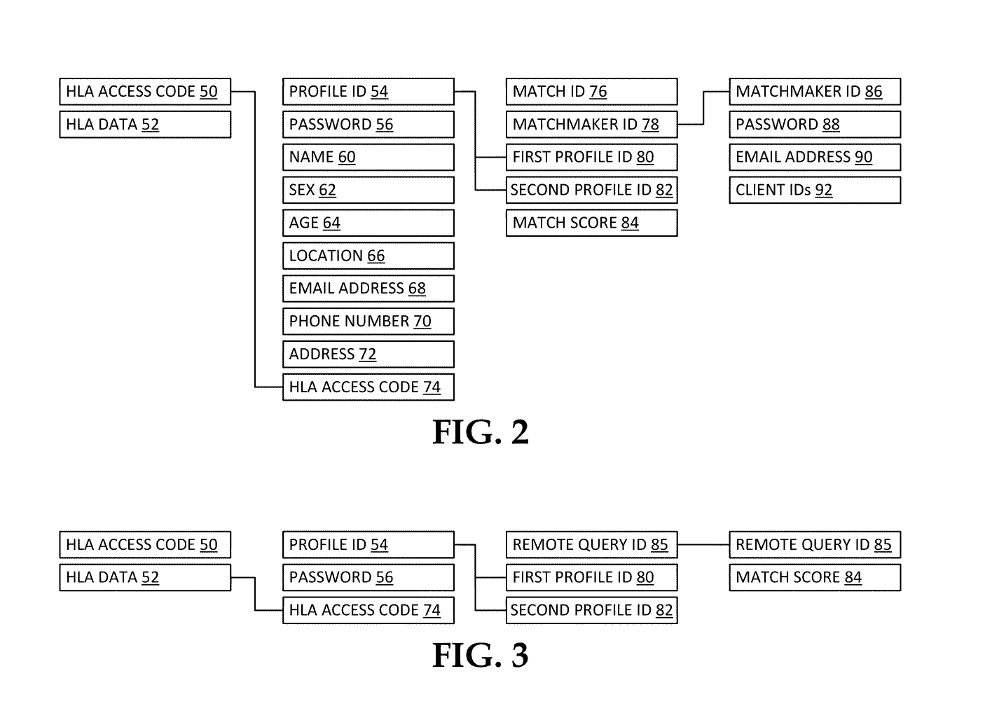

FIG. 2 shows an example data structure suitable for the server 26, particularly when matchmakers 38 are involved. The data structure can be implemented using database tables, queries, files, or similar. Lines connecting elements in FIG. 2 can be understood to represent relationships between sets of elements, such as local and foreign key relationships between database tables.

HLA information 52 for various different individuals is stored as indexed by unique access code 50, as discussed above. After DNA processing, the resultant HLA information 52 can be uploaded or entered by an administrator of the server 26.

Personal information about interested individuals, such as name 60, sex 62, age 64, and general location 66 (e.g., city, state/province, etc.) are stored indexed by profile ID 54. Photographs of individuals may also be stored. Access to individual profiles can be managed by credentials such as a password 56. Each profile can further store contact information for the interested persons, such as email address 68, phone number 70, and physical address 72. Each profile can further store a unique access code 74 for use as a foreign key for access to the HLA information 52. Accordingly, each interested person can access their profile to obtain their personal HLA information.

Match information can be stored as indexed by match ID 76. Each match or potential match is tied to an individual matchmaker ID 78 and identifies two profiles by way of first and second profile IDs 80, 82, which act as foreign keys. A match score 84, once determined, can be stored for each match.

Matchmakers can be provided with access to the data by way of matchmaker profiles indexed by matchmaker ID 86. Matchmaker profiles can be managed by credentials such as a password 88, and may store contact information for the matchmaker, such as an email address 90. Matchmaker ID 86 can be used as a foreign key to access matches and potential matches.

The server 26 can be configured to allow a user logged in as a matchmaker to create profiles for interested individuals, and such profiles may be tied to the matchmaker's account as a client. For this purpose, matchmaker profiles may further store a list of client IDs 92 that map to the profile IDs 54 of the interested persons.

The server 26 can be configured to allow a user logged in as a matchmaker to run queries on the profiles of interested persons, so as to form matches and potential matches. Such queries can make reference to the HLA information 52 stored for interested individuals, as will be discussed in further detail below. The result of such a query is an indication of compatibility that is inversely related to HLA similarly.

In some embodiments, the server 26 is configured to limit a matchmaker to running queries only on those profiles listed under that matchmaker's client IDs 92. In other embodiments, a matchmaker can run queries referencing one profile listed under that matchmaker's client IDs 92 and another profile irrespective of whether such profile is listed under that matchmaker's client IDs 92. In still further embodiments, no such limitations are placed on the profiles that may be selected by the matchmaker. In the latter two cases, the server 26 can be configured to prevent the matchmaker from obtaining the contact information of the interested persons whose profiles are not listed under that matchmaker's client IDs. Rather, the server 26 can forward contact inquiries for such an interested person to the respective matchmaker who has the respective profile listed under his/her client IDs 92.

The server 26 can further be configured to notify interested individuals by, for example, sending an email message when HLA information 52 has been processed and is available for viewing. Similarly, the server 26 can be configured to notify the respective matchmaker, again, for example, via email, when the HLA information 52 is available for performing comparisons with other interested individuals.

FIG. 3 shows an example data structure suitable for the server 26, particularly when external third-party services 40 are provided access. The data structure can be implemented using database tables, queries, files, or similar. Lines connecting elements in FIG. 3 can be understood to represent relationships between sets of elements, such as local and foreign key relationships between database tables. The description for FIG. 2 can be referenced for like-numbered elements.

In this data structure, profiles are reduced to a profile ID 54, password 56, and unique access code 74 for HLA information. Thus, the interested person can log in to view their own HLA information based on the access code provided with the swab kit.

Queries can be constructed to determine compatibility between two individuals represented by profiles IDs 80, 82. Such queries can be performed in response to a request received at the server 26 from a third-party service 40 that has knowledge of the profile IDs 80, 82 or other indicating information. The server 26 can track a query based on a remote query ID 85 associated with the profile IDs 80, 82 and respond with a match score 84 indicative of the degree of HLA dissimilarity of the individuals represented by the profile IDs 80, 82. For example, a third-party service 40 may make a request for a match score 84 for profiles IDs 80, 82, with the request accompanying a remote query ID 85, such as a hash or other relatively unique identifier. The server 26, in response, determines the match score 84 using the techniques described herein, and responds to the third-party service 40 with the match score 84 and the remote query ID 85. Accordingly, the third-party service 40 can be configured to accept input from interested individuals of their profile ID 54, as stored by the server 26, so that the third-party service 40 can generate and send such queries to the server 26.

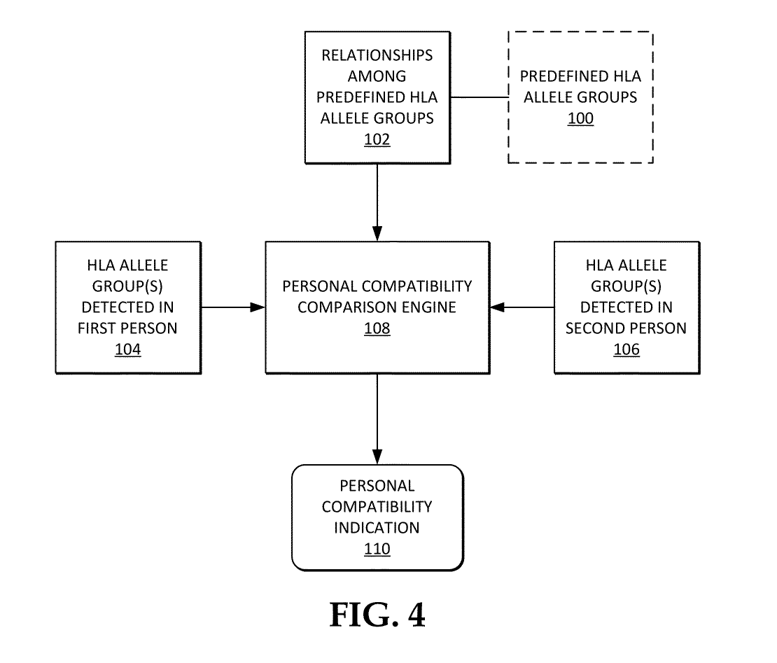

FIG. 4 illustrates data and operations at the server 26 for processing matches.

A predefined set of allele groups 100 for one or more HLA genes is defined to contain related alleles for each HLA gene considered. In some embodiments, one or more of the HLA-A, HLA-B, and HLA-DRB1 genes have allele groups defined by the set of allele groups 100. In other embodiments, all of the HLA-A, HLA-B, and HLA-DRB1 genes have allele groups defined by the set of allele groups 100. Each of the allele groups 100 can be assigned a name, number, or other identifier.

Relationships 102 for the predefined set of allele groups 100 can be stored in a database table or other data structure suitable to be updated and maintained based on additions or refinements made to the allele groups. The relationships 102 can be indexed by allele group identifier and can express a similarity between two (or more) allele groups 100. For example, a first allele group may be classified as similar to a second allele group, while the first allele group may be classified as dissimilar to a third allele group. The relationships 102 can express degrees of similarity using numerical values.

The allele groups present in two interested persons 18 (FIG. 1) are determined based on DNA extraction and probing 24. Primers are defined for each of group of the set of allele groups 100 and used to identify of which groups the alleles present in the first and second interested persons are members. That is, each of the allele groups 100 is defined by one or more primer sets, and when such one or more primer sets binds to DNA extracted from a person's DNA sample to amplify the corresponding allele, the person is considered to have the respective allele group. The primers can be single nucleotide polymorphism (SNP) primers designed to be specific for a minimum of one nucleotide difference between sequences. For each HLA gene considered, it is expected that each interested person will have two alleles, one for each copy of the gene that is carried, and that the two alleles will fall within one or two of the defined groups.

After the DNA extraction and probing 24, the identifiers of the HLA allele groups determined to be present in the first person 104 and the identifiers of the HLA allele groups determined to be present in the second person 106 can be inputted into the server 26 and stored in a suitable data structure.

The server 26 is configured to execute a personal compatibility comparison engine 108, which receives as input the HLA allele groups of the first person 104 and the HLA allele groups of the second person 106. The engine 108 references the relationships 102 among the predefined set of allele groups 100 to determine an overall dissimilarity. The engine 108, for each HLA gene considered, is configured to compare at least one first allele group present in the first person 104 to at least one second allele group present in the second person 106 to determine a dissimilarity of the first allele group to the second allele group. Referencing the relationships 102, the engine 108 calculates a similarity based on the comparison and outputs an indication 110 of personal compatibility of the first person and the second person, with the personal compatibility being inversely related to the similarity.

For example, if the first allele group and the second allele group are the same, the engine 108 determines a maximum similarity and the personal compatibility indication 110 accordingly indicates that the first and second persons are relatively incompatible. If the first allele group and the second allele group are indicated by the relationships 102 to be highly dissimilar, then a low similarity factor is determined and the personal compatibility indication 110 indicates that the first and second persons are relatively compatible. Degrees of HLA gene dissimilarity and directly related degrees of romantic compatibility are available based on a number of allele groups 100 defined and their relationships 102.

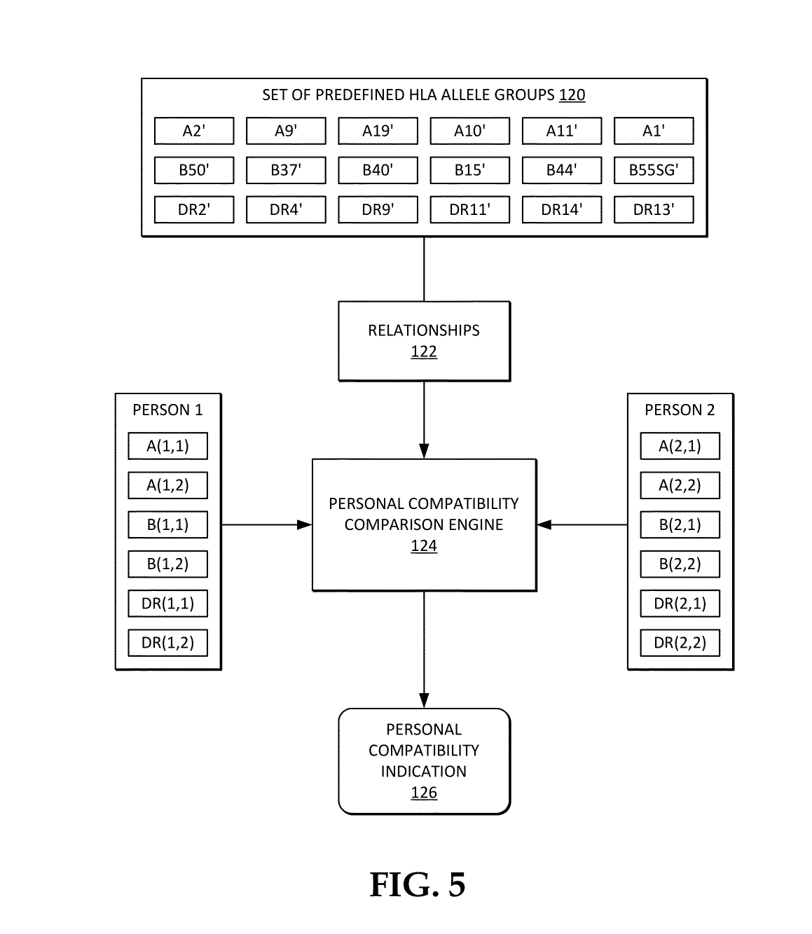

With reference to FIG. 5, a set of predefined allele groups 120 includes allele groups for all of the HLA-A, HLA-B, and HLA-DRB1 genes. In some embodiments, the set of allele groups 120 includes one or more of an A2′ group, an A9′ group, an A19′ group, an A10′ group, an A11′ group, and an A1′ group, which can be based on or the same as the respective known HLA-A2, HLA-A9, HLA-A19, HLA-A10, HLA-A11, and HLA-A1 serotypes. (Prime notation is used herein to illustrate that the known HLA definition of an allele group may be used, that a modified definition may be used, or that an entirely new definition may be used.) In some embodiments, the set of allele groups 120 includes one or more of a B50′ group, a B37′ group, a B40′ group, a B15′ group, a B44′ group, and a B55SG′ group, which can be based on or the same as the respective known HLA-B50′, HLA-B37′, HLA-B40′, HLA-B15′, HLA-B44′, and HLA-B55SG′ serotypes. In some embodiments, the set of allele groups 120 includes one or more of a DR2′ group, a DR4′ group, a DR9′ group, a DR11′ group, a DR14′ group, and a DR13′ group, which can be based on or the same as the respective known HLA-DR2′, HLA-DR4′, HLA-DR9′, HLA-DR11′, HLA-DR14′, and HLA-DR13′ serotypes. In some embodiments, two or more of the A2′, A9′, A19′, A10′, A11′, A1′, B50′, B37′, B40′, B15′, B44′, B55SG′, DR2′, DR4′, DR9′, DR11′, DR14′, and DR13′ allele groups are included in the set of allele groups 120. In some embodiments, all of the A2′, A9′, A19′, A10′, A11′, A1′, B50′, B37′, B40′, B15′, B44′, B55SG′, DR2′, DR4′, DR9′, DR11′, DR14′, and DR13′ allele groups are included in the set of allele groups 120.

In some embodiments, with reference to the attached sequence listing, the allele groups are defined by the following primers shown in Table 1, which can be provided in 6 wells of a polymerase chain reaction (PCR) primer array.

TABLE 1

| | | PRIMERS |
|---|---|---|
| GROUP | ALLELE GROUPS TARGETED | (ONE ROW PER WELL) |
| A2′ | a*02 | SEQ ID NO: 23 |
| | | SEQ ID NO: 24 |
| A9′ | a*32, a*24, a*23 | SEQ ID NO: 25 |
| | | SEQ ID NO: 26 |
| | | SEQ ID NO: 27 |
| | | SEQ ID NO: 28 |
| B40′ | b*40, b*41, b*47, b*27, | SEQ ID NO: 55 |
| | b*7, b*48, b*40 | SEQ ID NO: 56 |
| | | SEQ ID NO: 57 |
| B44′ | b*35, b*15, b*53, b*52, | SEQ ID NO: 68 |
| | b*51, b*49, b*44, b*48, | SEQ ID NO: 69 |
| | b*13 | SEQ ID NO: 70 |
| DR2′ | DRB1*15, DRB1*16 | SEQ ID NO: 94 |
| | | SEQ ID NO: 86 |
| DR4′ | DRB1*04 | SEQ ID NO: 95 |
| | | SEQ ID NO: 96 |
| | | SEQ ID NO: 97 |

In other embodiments, with reference to the attached sequence listing, the allele groups are defined by the following primers shown in Table 2, which can be provided in 18 wells of a PCR primer array.

TABLE 2

| | | PRIMERS |
|---|---|---|
| GROUP | ALLELE GROUPS TARGETED | (ONE ROW PER WELL) |
| A2′ | a*02 | SEQ ID NO: 23 |
| | | SEQ ID NO: 24 |
| A9′ | a*32, a*24, a*23 | SEQ ID NO: 29 |
| | | SEQ ID NO: 30 |
| | | SEQ ID NO: 31 |
| A19′ | a*03, a*33, a*31, a*29, | SEQ ID NO: 18 |
| | a*74 | SEQ ID NO: 19 |
| | | SEQ ID NO: 20 |
| | | SEQ ID NO: 21 |
| A10′ | a*25, a*66, a*26, a*34 | SEQ ID NO: 8 |
| | | SEQ ID NO: 9 |
| A11′ | a*11 | SEQ ID NO: 14 |
| | | SEQ ID NO: 15 |
| A1′ | a*01 | SEQ ID NO: 3 |
| | | SEQ ID NO: 4 |
| B50′ | b*45, b*50 | SEQ ID NO: 76 |
| | | SEQ ID NO: 77 |
| B37′ | b*37, b*39, b*14, b*67, | SEQ ID NO: 46 |
| | b*38, b*08, b*42 | SEQ ID NO: 47 |
| B40′ | b*40, b*41, b*47, b*27, | SEQ ID NO: 55 |
| | b*7, b*48, b*40 | SEQ ID NO: 56 |
| | | SEQ ID NO: 57 |
| B15′ | b*35, b*18, b*15 | SEQ ID NO: 36 |
| | | SEQ ID NO: 37 |
| B44′ | b*35, b*15, b*53, b*52, | SEQ ID NO: 68 |
| | b*51, b*49, b*44, b*48, | SEQ ID NO: 69 |
| | b*13 | SEQ ID NO: 70 |
| B55SG′ | b*56, b*35, b*55, b*78, | SEQ ID NO: 82 |
| | b*54, b*57, b*58 | SEQ ID NO: 83 |
| | | SEQ ID NO: 84 |
| DR2′ | DRB1*15, DRB1*16 | SEQ ID NO: 94 |
| | | SEQ ID NO: 86 |
| DR4′ | DRB1*04 | SEQ ID NO: 95 |
| | | SEQ ID NO: 96 |
| | | SEQ ID NO: 97 |
| DR9′ | DRB1*01, DRB1*07, | SEQ ID NO: 98 |
| | DRB1*09, DRB1*10 | SEQ ID NO: 99 |
| | | SEQ ID NO: 100 |
| | | SEQ ID NO: 101 |
| DR11′ | DRB1*11 | SEQ ID NO: 85 |
| | | SEQ ID NO: 86 |
| DR14′ | DRB1*03, DRB1*14, | SEQ ID NO: 89 |
| | DRB1*08, DRB1*12 | SEQ ID NO: 90 |
| | | SEQ ID NO: 91 |
| | | SEQ ID NO: 92 |
| | | SEQ ID NO: 93 |
| DR13′ | DRB1*13 | SEQ ID NO: 87 |
| | | SEQ ID NO: 88 |

In still other embodiments, with reference to the attached sequence listing, the allele groups are defined by the following primers shown in Table 3, which can be provided in 45 wells of a PCR primer array.

TABLE 3

| | | PRIMERS |
|---|---|---|
| GROUP | ALLELE GROUPS TARGETED | (ONE ROW PER WELL) |
| A2′ | a*02 | SEQ ID NO: 23 |
| | | SEQ ID NO: 24 |
| A9′ | a*32, a*24, a*23 | SEQ ID NO: 25 |
| | | SEQ ID NO: 26 |
| | | SEQ ID NO: 27 |
| | | SEQ ID NO: 28 |
| A9′ | a*32, a*24, a*23 | SEQ ID NO: 29 |
| | | SEQ ID NO: 30 |
| | | SEQ ID NO: 31 |
| A19′ | a*03, a*33, a*30, a*31, | SEQ ID NO: 16 |
| | a*29, a*74 | SEQ ID NO: 17 |
| A19′ | a*03, a*33, a*31, a*29, | SEQ ID NO: 18 |
| | a*74 | SEQ ID NO: 19 |
| | | SEQ ID NO: 20 |
| | | SEQ ID NO: 21 |
| A19′ | a*31, a*30 | SEQ ID NO: 22 |
| | | SEQ ID NO: 21 |
| A10′ | a*25, a*66, a*68, a*26, | SEQ ID NO: 5 |
| | a*34 | SEQ ID NO: 6 |
| | | SEQ ID NO: 7 |
| A10′ | a*25, a*66, a*26, a*34 | SEQ ID NO: 8 |
| | | SEQ ID NO: 9 |
| A10′ | a*68 | SEQ ID NO: 10 |
| | | SEQ ID NO: 11 |
| A11′ | a*11 | SEQ ID NO: 12 |
| | | SEQ ID NO: 13 |
| A11′ | a*11 | SEQ ID NO: 14 |
| | | SEQ ID NO: 15 |
| A1′ | a*01 | SEQ ID NO: 1 |
| | | SEQ ID NO: 2 |
| A1′ | a*01 | SEQ ID NO: 3 |
| | | SEQ ID NO: 4 |
| B50′ | b*56, b*15 | SEQ ID NO: 71 |
| | | SEQ ID NO: 72 |
| B50′ | b*45, b*50 | SEQ ID NO: 58 |
| | | SEQ ID NO: 73 |
| B50′ | b*15 | SEQ ID NO: 74 |
| | | SEQ ID NO: 75 |
| B50′ | b*45, b*50 | SEQ ID NO: 76 |
| | | SEQ ID NO: 77 |
| B37′ | b*37 | SEQ ID NO: 38 |
| | | SEQ ID NO: 39 |
| B37′ | b*08, b*42 | SEQ ID NO: 40 |
| | | SEQ ID NO: 41 |
| B37′ | b*38 | SEQ ID NO: 42 |
| | | SEQ ID NO: 43 |
| B37′ | b*39, b*14, b*67 | SEQ ID NO: 44 |
| | | SEQ ID NO: 45 |
| B37′ | b*37, b*39, b*14, b*67, | SEQ ID NO: 46 |
| | b*38, b*08, b*42 | SEQ ID NO: 47 |
| B37′ | b*37 | SEQ ID NO: 38 |
| | | SEQ ID NO: 41 |
| B37′ | b*39, b*14, b*67 | SEQ ID NO: 44 |
| | | SEQ ID NO: 43 |
| B40′ | b*27 | SEQ ID NO: 48 |
| | | SEQ ID NO: 49 |
| B40′ | b*40, b*41, b*47 | SEQ ID NO: 50 |
| | | SEQ ID NO: 51 |
| B40′ | b*7, b*48, b*40 | SEQ ID NO: 52 |
| | | SEQ ID NO: 53 |
| | | SEQ ID NO: 54 |
| B40′ | b*40, b*41, b*47, b*27, | SEQ ID NO: 55 |
| | b*7, b*48, b*40 | SEQ ID NO: 56 |
| | | SEQ ID NO: 57 |
| B40′ | b*27 | SEQ ID NO: 58 |
| | | SEQ ID NO: 49 |
| B15′ | b*15 | SEQ ID NO: 32 |
| | | SEQ ID NO: 33 |
| B15′ | b*35, b*18 | SEQ ID NO: 34 |
| | | SEQ ID NO: 35 |
| B15′ | b*35, b*18, b*15 | SEQ ID NO: 36 |
| | | SEQ ID NO: 37 |
| B44′ | b*44, b*48 | SEQ ID NO: 59 |
| | | SEQ ID NO: 60 |
| | | SEQ ID NO: 61 |
| B44′ | b*13 | SEQ ID NO: 62 |
| | | SEQ ID NO: 63 |
| B44′ | b*35, b*15, b*53, b*52, | SEQ ID NO: 64 |
| | b*51, b*49 | SEQ ID NO: 65 |
| | | SEQ ID NO: 66 |
| | | SEQ ID NO: 67 |
| B44′ | b*35, b*15, b*53, b*52, | SEQ ID NO: 68 |
| | b*51, b*49, b*44, b*48, | SEQ ID NO: 69 |
| | b*13 | SEQ ID NO: 70 |
| B55SG′ | b*57, b*58 | SEQ ID NO: 78 |
| | | SEQ ID NO: 79 |
| B55SG′ | b*56, b*35, b*55, b*78, | SEQ ID NO: 80 |
| | b*54 | SEQ ID NO: 81 |
| B55SG′ | b*56, b*35, b*55, b*78, | SEQ ID NO: 82 |
| | b*54, b*57, b*58 | SEQ ID NO: 83 |
| | | SEQ ID NO: 84 |
| DR2′ | DRB1*15, DRB1*16 | SEQ ID NO: 94 |
| | | SEQ ID NO: 86 |
| DR4′ | DRB1*04 | SEQ ID NO: 95 |
| | | SEQ ID NO: 96 |
| | | SEQ ID NO: 97 |
| DR9′ | DRB1*01, DRB1*07, | SEQ ID NO: 98 |
| | DRB1*09, DRB1*10 | SEQ ID NO: 99 |
| | | SEQ ID NO: 100 |
| | | SEQ ID NO: 101 |
| DR11′ | DRB1*11 | SEQ ID NO: 85 |
| | | SEQ ID NO: 86 |
| DR14′ | DRB1*03, DRB1*14, | SEQ ID NO: 89 |
| | DRB1*08, DRB1*12 | SEQ ID NO: 90 |
| | | SEQ ID NO: 91 |
| | | SEQ ID NO: 92 |
| | | SEQ ID NO: 93 |
| DR13′ | DRB1*13 | SEQ ID NO: 87 |
| | | SEQ ID NO: 88 |

In Tables 1, 2, and 3 above, each row represents a primer set that can be provided in wells of a primer array, such as a primer plate. In other embodiments, one or more of the primer sets listed (Table 1, 2, or 3) for each of the HLA-A, HLA-B, and HLA-DRB1 genes are provided in a primer array. In other embodiments, two or more of the primer sets listed (Table 1, 2, or 3) for each of the HLA-A, HLA-B, and HLA-DRB1 genes are provided in a primer array. In other embodiments, substantially all of the listed primer sets (Table 1, 2, or 3) are provided, where “substantially” can be taken to mean that one or more of the primer sets may be omitted provided that the calculations discussed herein are modified to compensate. This can be achieved in a manner similar to compensation for missing information, as also discussed herein. Generally, the more of the listed primer sets (Table 1, 2, or 3) that are used, the more accurate the results. However, omitting some of the primer sets does not make the results invalid. In still other embodiments, all of the listed primer sets (Table 1, 2, or 3) are provided in wells of a primer array. In any of these embodiments, additional primer sets may be added to additional wells.

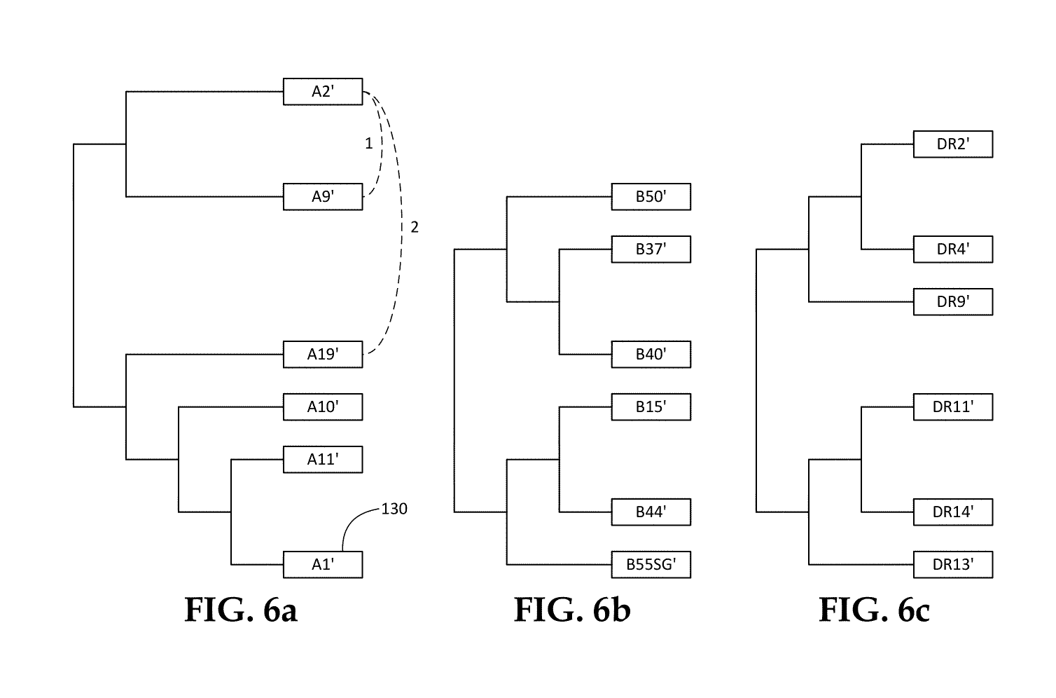

The relationships 122 are similar to the relationships 102 discussed above. Further, the relationships 122 are illustrated by the condensed phylogenetic trees shown in FIGS. 6a-6c. FIG. 6a shows the tree for allele groups A2′, A9′, A19′, A10′, A11′, and A1′. FIG. 6b shows the tree for allele groups B50′, B37′, B40′, B15′, B44′, and B55SG′. FIG. 6c shows the tree for allele groups DR2′, DR4′, DR9′, DR11′, DR14′, and DR13′.

Similarity values for allele groups can be determined based on a number of potential combinations for allele groups with reference to a number of hops between two leaf nodes 130 on a particular tree. That is, with reference to FIG. 6a, there are six possible combinations (irrespective of order) of two allele groups that are the same (e.g., A2′-A2′, A9′-A9′, etc.) and hence the similarity value for any two same allele groups can be taken as this number of combinations, namely, 6. Similarly, there are five possible combinations of two allele groups that are one hop removed (e.g., A2′-A9′, A9′-A19′, A19′-A10′, A10′-A11′, and A11′-A1′), with example hops being illustrated in the figures in dashed line. Hence, the similarity value for any two allele groups that are one hop removed can be taken as 5. Likewise, there are four possible combinations of two allele groups that are two hops removed (e.g., A2′-A19′, A9′-A10′, A19′-A11′, and A10′-A1′) and the similarity value for any two allele groups that are two hops removed can be taken as 4. There are three possible combinations of two allele groups that are three hops removed (e.g., A2′-A10′, A9′-A11′, and A19′-A1′) and the similarity value for any two allele groups that are three hops removed can be taken as 3. There are two possible combinations of two allele groups that are four hops removed (e.g., A2′-A11′ and A9′-A1′) and the similarity value for any two allele groups that are four hops removed can be taken as 2. And finally, there is one possible combination of two allele groups that are five hops removed (e.g., A2′-A1′) and the similarity value for any two allele groups that are five hops removed can be taken as 1. In addition, it should be apparent that the total number of possible combinations is the sum of the above numbers, which in this example is 21. The same logic applies to determining similarity values for the HLA-B (FIG. 6b) and HLA-DRB1 (FIG. 6c) genes.

In other words, and to further illustrate the above logic, the number of hops between two leaf nodes 130 on a particular tree can be taken as a dissimilarity value for the two allele groups represented by the two leaf nodes 130. For example, as shown in dashed lines on FIG. 6a, allele groups A2′ and A9′ are adjacent and thus one hop there-between results in a dissimilarity value of 1, allele groups A2′ and A19′ are separated by two hops and thus have a dissimilarity value of 2, and so on for each combination of two allele groups. For each combination of two allele groups, a similarity value can be calculated by subtracting the respective dissimilarity value from the total number of allele groups (or leaf nodes 130). In the example shown, allele groups A2′ and A1′ have a dissimilarity value of 5 and thus have a similarity value of 1 (i.e., 6 allele groups for HLA-A minus the dissimilarity value of 5). The same applies to the HLA-B and HLA-DRB1 condensed phylogenetic trees of FIGS. 6b and 6c.

It should be understood that similarity and dissimilarity are inversely related and determining a similarity is equivalent to determining a dissimilarity, provided that each are identified as such to the end consumer.

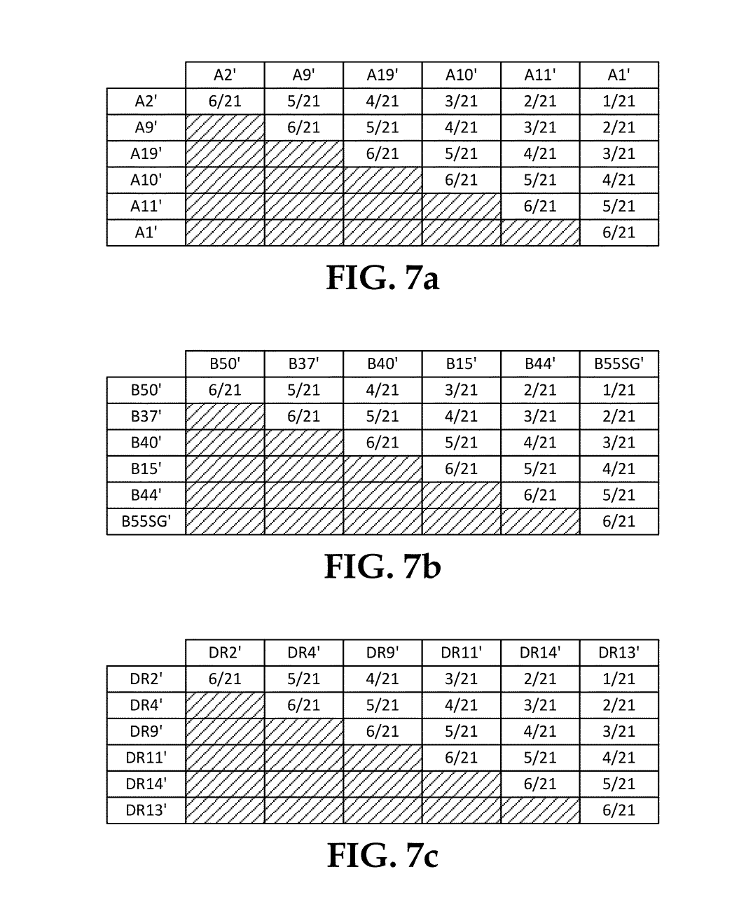

The relationships 122 can be numerically defined by tables of predefined similarity factors, as shown in FIGS. 7a-7c. FIG. 7a shows similarity factors for each possible combination of allele groups for the HLA-A gene. Columns indicate an allele group determined to be present in one of the interested persons, and rows indicate an allele group determined to be present in the other of the interested persons. The similarity factors in the cells are fractional values with similarity value in the numerator and a total number of possible combinations in the denominator. In the case of the six HLA-A groups discussed above, the total number of possible combinations is 21 noting that order is unimportant and repetition is permitted (i.e., two people may have alleles of the same group). Thus, the similarity factors represent a normalized relatedness of two alleles from the two interested persons. For example, if the first copy of the HLA-A gene in the first person is of the A2′ allele group and the first copy of the HLA-A gene in the second person is of the same group, then the similarity factor for those two alleles is 6/21. If, on the other hand, the first copy of the HLA-A gene in the second person is of the A19′ group, then the similarity factor is 4/21. The same applies to the HLA-B and HLA-DRB1 tables of FIGS. 7b and 7c.

Referring back to FIG. 5, with the relationships 122 defined as above, the allele groups detected in each of the two persons can be used as inputs to a personal compatibility comparison engine 124 that references the relationships 122, which can include looking up values from the tables of FIGS. 7a-7c. That is, each copy of the first person's HLA-A genes are determined to belong to groups A(1,1) and A(1,2), where the first index represents the person and the second index indicates the gene copy. Likewise, the first person's HLA-B genes are determined to belong to groups B(1,1) and B(1,2), and so on for all elements of A( ) and B( ), with the same logic continuing and thus the second person's HLA-DRB1 genes determined to be of groups DR(2,1) and DR(2,2).

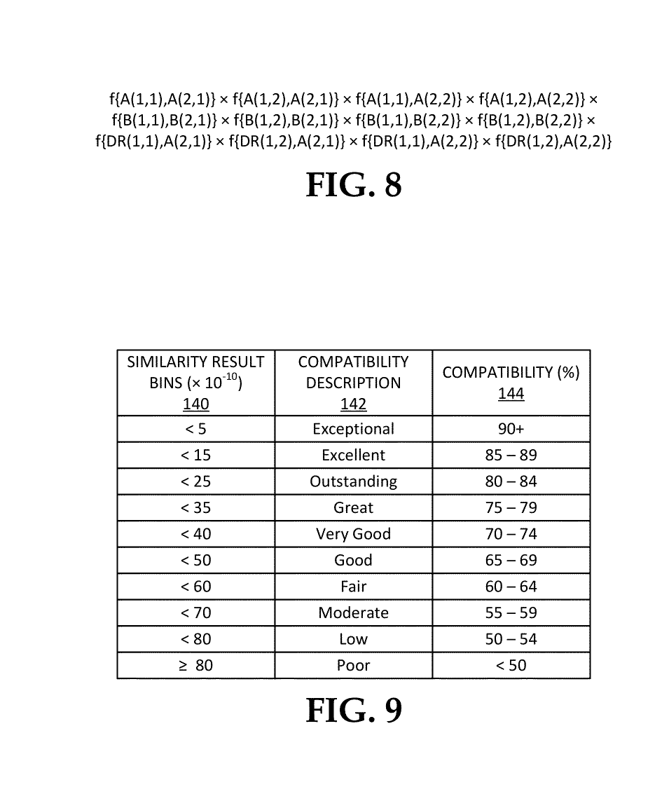

The personal compatibility comparison engine 124 can be configured to lookup similarity factors, using for example the tables of FIGS. 7a-7c, for each combination of alleles of a particular HLA gene between the two interested persons being compared. That is, for the HLA-A gene, the first allele in the first person A(1,1) and the first allele in the second person A(2,1) are used to determine a similarity value from the table of FIG. 7a. Likewise, the second allele in the first person A(1,2) and the first allele in the second person A(2,1) are used to determine a similarity value from the table of FIG. 7a, and so on, until a similarity value has been determined for each of the four possible combinations: A(1,1) to A(2,1), A(1,2) to A(2,1), A(1,1) to A(2,2), and A(1,2) to A(2,2). The same is performed for the HLA-B and HLA-DRB1 genes. When each of the three HLA genes is defined to have six groups, as mentioned above, a total of 12 comparisons (four for each gene) are made and twelve similarity factors result. All of the similarity factors can then be multiplied together to obtain a combined similarity result indicative of a total HLA comparison of the two individuals. Where a function, f, is defined to perform a value lookup in the respective table (FIGS. 7a-7c) based on two inputted allele groups (one from each person), FIG. 8 illustrates an example calculation of the combined similarity result.

The combined similarity result represents a probability of two people with that similarity or dissimilarity coming together. A lower probability equates to greater dissimilarity and thus a higher compatibility.

One advantage of the above calculation is that HLA relatedness is quantified in a way that allows for finer-grained analysis. Not only does the process detect the presence of certain allele groups within two interested persons, the process further determines a numerical relatedness based on combinational comparisons of the allele groups.

The personal compatibility comparison engine 124 can be further configured to reference indications of compatibility with reference to a calculated combined similarity result. FIG. 9 illustrates a table of compatibility descriptions 142 and percentage ranges 144 based on similarity factor bins 140. As can be seen, romantic compatibility increases with decreasing HLA similarity factor. The combined similarity result for two interested persons, as determined by the engine 124, can be used as a key to determine the similarity factor bin 140 and thus the compatibility description 142 and percentage 144 for that couple.

Referring back to FIG. 5, one or more of the determined compatibility description 142 and percentage 144 can be output by the engine 124 as a personal compatibility indication 126. The personal compatibility indication 126 can then be communicated to the matchmaker 38 or third-party service 40 (FIG. 1).

In an example, suppose it is determined that a first individual, James, has HLA genes having alleles that fall into groups A9′, A19′, B40′, B44′, DR2′, and DR13′, and a second individual, Barbara, as HLA genes of groups A2′, A1′, B37′, B40′, DR11′, and DR14′. Referencing the table of FIG. 7a, A9′ and A2′ have a similarity factor of 5/21, A9′ and A1′ have 2/21, A19′ and A2′ have 4/21, and A19′ and A1′ have 3/21. Referencing the table of FIG. 7b, B40′ and B37′ have a similarity factor of 5/21, B40′ and B40′ have 6/21, B44′ and B37′ have 3/21, and B44′ and B40′ have 4/21. Referencing the table of FIG. 7c, DR2′ and DR11′ have a similarity factor of 3/21, DR2′ and DR14′ have 2/21, DR13′ and DR11′ have 4/21, and DR13′ and DR14′ have 5/21. The combined similarity result is then determined by the product (FIG. 8) of (5/21)*(2/21)*(4/21)*(3/21)*(5/21)*(6/21)*(3/21)*(4/21)*(3/21)*(2/21)*(4/21)*(5/21), which approximately equals 7.0E-10. The combined similarity result is then applied to the table of FIG. 9 to determine that James' and Barbara's compatibility is “Excellent” with a score of between 85 and 89.

The personal compatibility comparison engine 124 can also be configured to accommodate one or more missing allele groups from the two interested persons, which may result from lab errors, poor sample quality, or if the person carries an allele that is not detected. For each missing allele group, the engine 124 performs bounding calculations using the lowest possible values and the highest possible values for the missing allele group. The engine 124 can perform a separate calculation for each assumption for the group of the missing allele and take the largest and smallest values as upper and lower bounds. In the above example, suppose that Barbara's A2′ allele is missing. The engine 124 compares A9′ and A19′ to assumed identities of the missing allele and determines that the maximum similarity occurs when the missing allele is A9′ or A19′ (with similarity factors of 6/21 and 5/21) and the minimum similarity occurs when the missing allele is A1′ (with similarity factors of 2/21 and 3/21). Thus, two combined similarity results are determined using the equation of FIG. 8, one using 6/21 and 5/21 and another using 2/21 and 3/21. The resulting combined similarity results of 10.6E-10 and 2.1E-10 can be averaged and then used to determine a corresponding compatibility (“Excellent” with a score of 85-89), or can be used to determine a corresponding compatibility range of “Excellent to Exceptional” with a score range of 85 to 90+. Compensating for missing DNA information can advantageously reduce the need to retake or reprocess DNA samples, which can be costly and time consuming.

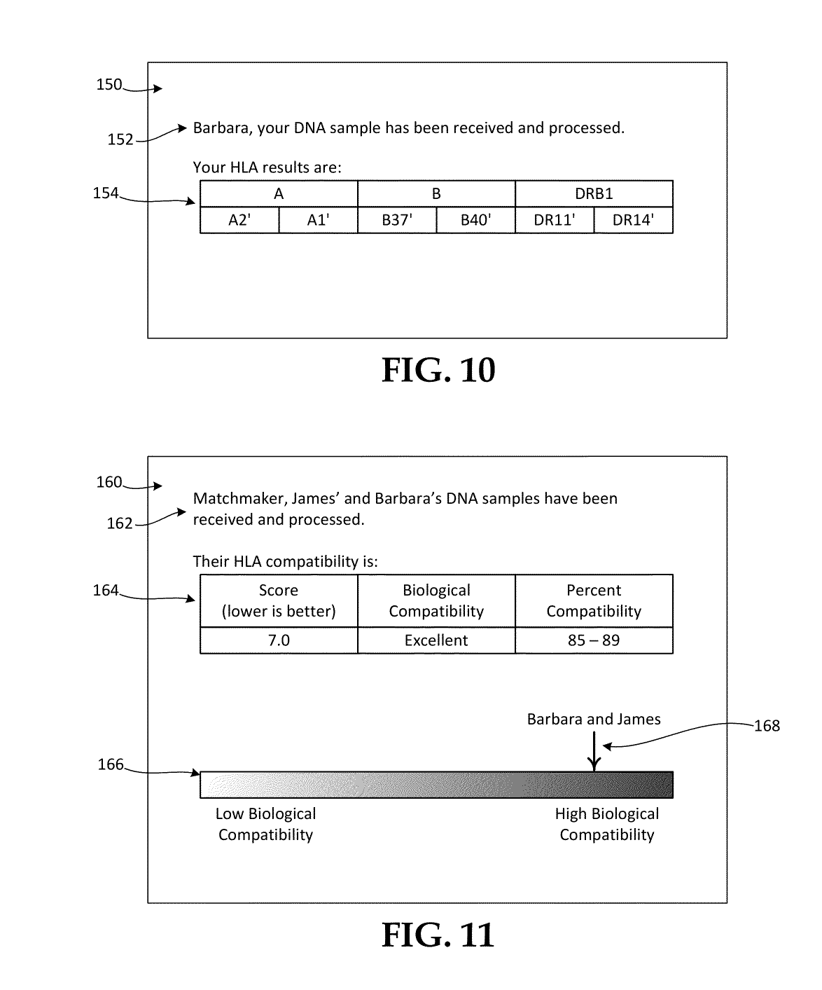

FIG. 10 shows example output of HLA information 28 (FIG. 1) for an individual interested person 18. A web page 150 or output of another format includes appropriate descriptive text 152 and a table 154 of HLA results containing the detected allele groups for each gene examined.

FIG. 11 shows example output of a personal compatibility indication 36 (FIG. 1) for two interested persons 18, who may have each contacted a matchmaker. A web page 160 or output of another format includes appropriate descriptive text 162 and a table 164 containing one or more indications of compatibility, such as the determined combined similarity result (shown as a “score” and trimmed of exponent), a compatibility description 142 (FIG. 9), and a compatibility percentage 144. A graphical indicator 166 may, alternatively or additionally, be provided as an indication of compatibility. In some embodiments, the graphical indicator 166 includes a linear bar on which the combined similarity result is plotted, and as such, the indicator 166 may include a plot point 168, such as an arrow, and may have coloring to emphasise personal compatibility or lack thereof (e.g., a red to blue gradient).

With reference to FIG. 1, the DNA extraction and probing 24 can be performed by the following process. The laboratory receives a DNA sample 20 from the interested individual or the respective matchmaker. DNA is extracted from the swab containing the DNA sample 20. An extraction kit, such as Life Technologies PureLink Genomic DNA kits (Catalogue number: K1820-02) can be used. If extraction fails, then another swab, if available, can be processed.

To amplify the HLA genes of interest, which are discussed above, two 20 microliter (ul) PCRs can be run on the extracted DNA. The same settings may be used for both reactions, so that the reactions can be run at the same time. The following materials may be used: Taq DNA polymerase (recombinant; catalogue number: 10342-020) available from Invitrogen by Life Technologies, 10 mM dNTP mix (PCR grade; catalogue number: 18427-088) available from Invitrogen by Life Technologies, and primers to target the HLA genes of interest, available from Sigma-Aldrich and diluted to a stock concentration of 100 uM and a working stock of 10 uM. Stocks of the PCR buffer are then made (4 or 5 aliquots, stored at −20 C) as follows: 10×PCR buffer (100 ul), dNTP mixture (20 ul), MgCl2 (30 ul), and Millipore H2O (725 ul). Two PCR reactions may then be set up, each as follows: stock of PCR buffer mixture (17.5 ul), primer mix (1 ul), DNA (1 ul), and Taq (0.5 ul). The PCR reactions may then be run with the following settings, shown in Table 4.

TABLE 4

| | | | |
|---|---|---|---|
| Repeat 1X | Denaturation | 94 C. | 3 minutes |
| Repeat 40X | Denature | 94 C. | 45 seconds |
| | Anneal | 65 C. | 30 seconds |
| | Extension | 72 C. | 1 minute |
| Repeat 1X | Incubate | 72 C. | 10 minutes |
| Hold | Hold | 4 C. | infinity |

To determine which allele groups are present in the DNA sample, two 20 ul quantitative real-time (qRT) PCR reactions using SYBR green a well plate with embedded primers for the allele groups, as discussed above. The well plate can be primed with multiple instances of each primer, so that one well plate can be used for multiple samples, such as samples from two interested persons whose compatibility is being determined. When the matchmaker (or interested person) sends to the laboratory samples from two interested persons in the same package, use of the well plate in this manner can help reduce the chance that results will be mixed up. A 96-well plate can be used, such as the type available from Bio-Rad. Additional materials include: iQSYBR Green Supermix (catalogue number: 170-8885) and DNA from both PCR reactions and from genomic extracted DNA. The PCR products can be diluted 1:20 before being added to the qRT reaction. Per reaction, the following volumes shown in Table 5 may be used.

TABLE 5

| Volumes per reaction | PCR product 1 | PCR product 2 | Genomic DNA |
|---|---|---|---|
| Millipore H2O | 9 ul | 9 ul | 9.5 ul |
| Template | 1 ul | 1 ul | 0.5 ul |
| SYBR | 10 ul | 10 ul | 10 ul |

The qRT-PCR reactions may then be run with the following settings, shown in Table 6.

TABLE 6

| | | | |
|---|---|---|---|
| Repeat 1X | Denaturation | 95 C. | 3 minutes |
| Repeat 40X | Denature | 95 C. | 15 seconds |
| | Anneal | 63.5 C. | 30 seconds |
| | Extension | 72 C. | 30 seconds |
| Repeat 1X | Melt Curve | 55 C.-95 C. | Read every 0.5 C. |
| | Incremental | | |
| Hold | Hold | 4 C. | infinity |

It will be understood by one of ordinary skill in the art upon reading this disclosure that any of the volumes, temperature, times, and other quantities discussed herein can be modified to accommodate different yet equivalent protocols. Moreover, suitable substitutes for specified equipment and material will also be apparent to such person in light of this disclosure, and the specified equipment and material is not intended to be limiting.

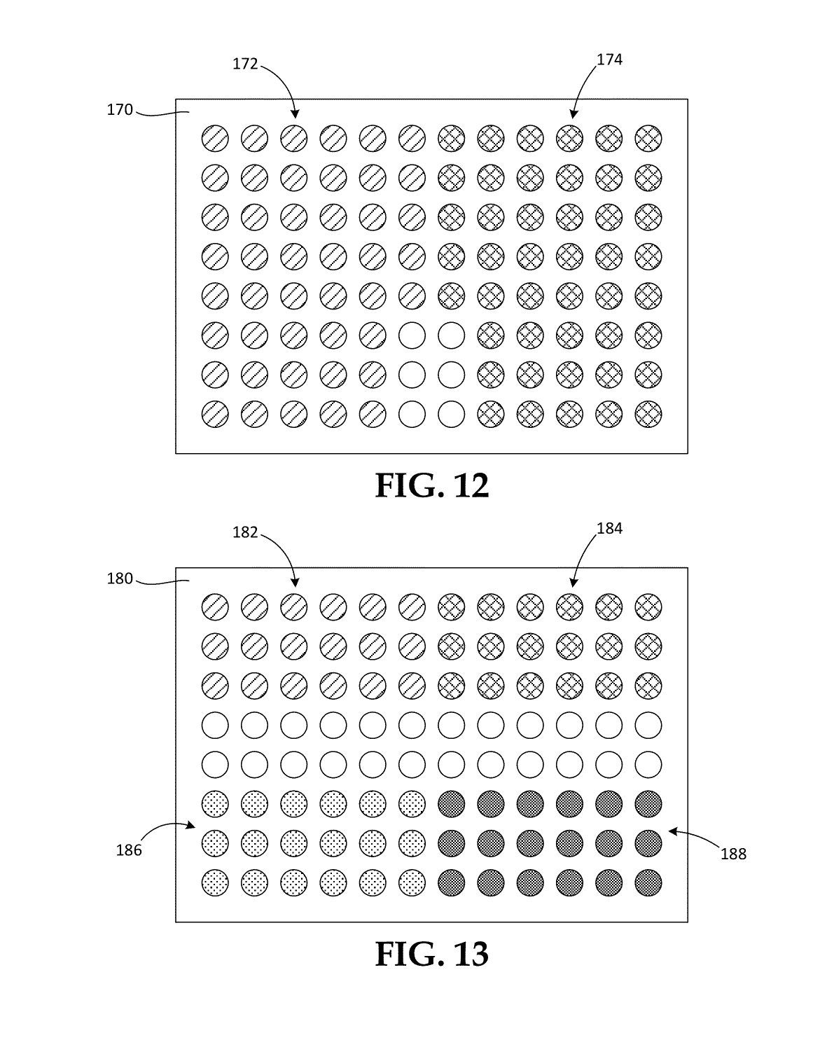

With reference to FIG. 12 a PCR primer plate 170 is shown. The primer plate 170 is an example of a suitable PCR primer array. The primer plate 170 has 96 wells divided into two regions 172, 174. The first region 172 contains primers for analysis of a first interested person's HLA genes in 45 wells. The second region 174 contains primers for analysis of a second interested person's HLA genes in 45 wells. The primer sets may be assigned based on Table 3, above, with one well of each region 172, 174 containing the primers listed in one row of the table.

With reference to FIG. 13 another PCR primer plate 180 is shown. The primer plate 180 is an example of a suitable PCR primer array. The primer plate 180 has 96 wells divided into four regions 182-188. The first region 182 contains primers for analysis of a first interested person's HLA genes in 18 wells. The second region 184 contains primers for analysis of a second interested person's HLA genes in 18 wells. The third region 186 contains primers for analysis of a third interested person's HLA genes in 18 wells. The fourth region 188 contains primers for analysis of a fourth interested person's HLA genes in 18 wells. The primer sets may be assigned based on Table 2, above, with one well of each region 182-188 containing the primers listed in one row of the table.

Other primer array configurations are also contemplated. For example, a 96-well primer plate can be used for 16 groupings of six primer sets assigned based on Table 1, above. This can allow batched analysis for 16 interested individuals, such as eight potential couples. In another example, a 384-well primer plate is used.

The PCR primer arrays discussed herein, such as the primer plates 170, 180, are not limited by geometry or materials and can have a rectangular shape, a disc shape, a linear strip of tubes, a collection of loose tubes, or other structure. In addition, the wells need not be limited to any particular structure or layout, and the figures merely depict examples.

The allele groups discussed herein are examples. More or fewer groups can be used and group definitions can be modified, without departing from the scope of the invention. Sequences can be obtained from an IMGT/HLA Database (http://www.ebi.ac.uk/ipd/imgt/hla/align.html), with exons 2 and 3 being suitable for HLA-A and HLA-B and exon 2 being suitable for HLA-DRB1. It may be desirable to select these particular regions because they are some of the more diverse regions of the HLA genes, and further are relatively small and amenable to quantitative real-time polymerase chain reaction (qRT-PCR) processing. Sequences can be formatted and aligned using a tool such as the Mafft multiple sequence alignment program (http://mafft.cbrc.jp/alignment/server/index.html). Phylogenetic analysis of the sequences, including bootstrapping of the alignment to increase the robustness of the phylogenetic tree, can be performed using a program such as Clustal X2 (http://www.clustal.org/clustal2/). Phylogenetic trees, such as the condensed trees discussed herein, can be created using a tool such as NJplot (http://pbil.univ-lyon1.fr/software/njplot.html), and serological groups may be referenced. Finally, alleles can be segregated into groups using Jalview (http://www.jalview.org/), which can aid visualization of sequences for primer design.

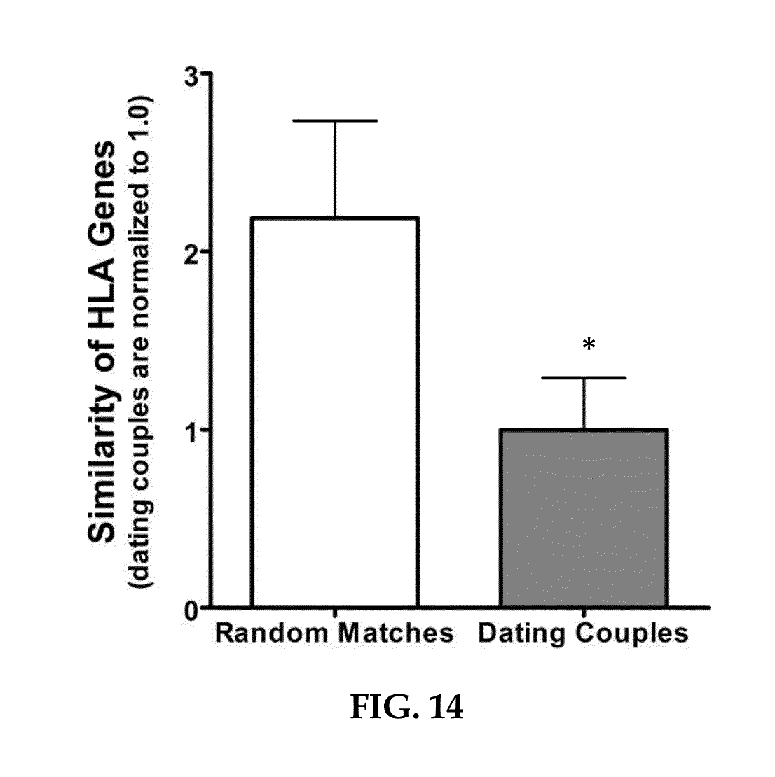

An alpha test was performed using a sample size of 65 individuals. Approximately half of the 65 individuals (15 couples) reported to be in relationships, while the remainder identified as single. Using the teachings disclosed herein, the similarity of HLA alleles between couples and singles randomly assorted into theoretical couples was determined. As illustrated in FIG. 14, normalized results showed that randomly matched pairs had a 2-fold greater similarity in their HLA genes than actual dating couples.

Advantages of the above techniques for matchmakers or third-party services, such as dating companies, may include higher success rates, more satisfied customers, increased clientele, and a competitive advantage. Advantages of the above techniques for individuals may include increased sense of physical attraction with one's partner, a more satisfying sex life, healthier children, increased fertility rates, and a higher likelihood of long-term relationship.

While the foregoing provides certain non-limiting example embodiments, it should be understood that combinations, subsets, and variations of the foregoing are contemplated. The monopoly sought is defined by the claims.

What is claimed is:

1. A primer array for use in analyzing personal compatibility based on human leukocyte antigen (HLA) genes, the primer array comprising:
at least one HLA-A primer set selected to target allele groups of an HLA-A gene of a DNA sample to undergo quantitative real-time polymerase chain reaction (qRT-PCR) processing;
at least one HLA-B primer set selected to target allele groups of an HLA-B gene of the DNA sample to undergo qRT-PCR processing; and
at least one HLA-DRB1 primer set selected to target allele groups of an HLA-DRB1 gene of the DNA sample to undergo qRT-PCR processing.

2. The primer array of claim 1 comprising:
two or more HLA-A primer sets selected from a first group consisting of:
a primer set having primers of the following sequences: SEQ ID NO: 23 and SEQ ID NO: 24;
a primer set having primers of the following sequences: SEQ ID NO: 29, SEQ ID NO: 30, and SEQ ID NO: 31;
a primer set having primers of the following sequences: SEQ ID NO: 18, SEQ ID NO: 19, SEQ ID NO: 20, and SEQ ID NO: 21;
a primer set having primers of the following sequences: SEQ ID NO: 8 and SEQ ID NO: 9;
a primer set having primers of the following sequences: SEQ ID NO: 14 and SEQ ID NO: 15; and
a primer set having primers of the following sequences: SEQ ID NO: 3 and SEQ ID NO: 4;
two or more HLA-B primer sets selected from a second group consisting of:
a primer set having primers of the following sequences: SEQ ID NO: 76 and SEQ ID NO: 77;
a primer set having primers of the following sequences: SEQ ID NO: 46 and SEQ ID NO: 47;
a primer set having primers of the following sequences: SEQ ID NO: 55, SEQ ID NO: 56, and SEQ ID NO: 57;
a primer set having primers of the following sequences: SEQ ID NO: 36 and SEQ ID NO: 37;
a primer set having primers of the following sequences: SEQ ID NO: 68, SEQ ID NO: 69, and SEQ ID NO: 70; and
a primer set having primers of the following sequences: SEQ ID NO: 82, SEQ ID NO: 83, and SEQ ID NO: 84;
two or more HLA-DRB1 primer sets selected from a third group consisting of:
a primer set having primers of the following sequences: SEQ ID NO: 94 and SEQ ID NO: 86;
a primer set having primers of the following sequences: SEQ ID NO: 95, SEQ ID NO: 96, and SEQ ID NO: 97;
a primer set having primers of the following sequences: SEQ ID NO: 98, SEQ ID NO: 99, SEQ ID NO: 100, and SEQ ID NO: 101;
a primer set having primers of the following sequences: SEQ ID NO: 85 and SEQ ID NO: 86;
a primer set having primers of the following sequences: SEQ ID NO: 89, SEQ ID NO: 90, SEQ ID NO: 91, SEQ ID NO: 92, and SEQ ID NO: 93; and
a primer set having primers of the following sequences: SEQ ID NO: 87 and SEQ ID NO: 88.

3. The primer array of claim 2, comprising substantially all of the HLA-A primer sets of the first group, substantially all of the HLA-B primer sets of the second group, and substantially all of the HLA-DRB1 primer sets of the third group.

4. The primer array of claim 2, comprising two identical groupings of the two or more HLA-A primer sets of the first group, the two or more HLA-B primer sets of the second group, and the two or more HLA-DRB1 primer sets of the third group.

5. The primer array of claim 2, comprising four identical groupings of the two or more HLA-A primer sets of the first group, the two or more HLA-B primer sets of the second group, and the two or more HLA-DRB1 primer sets of the third group.

6. The primer array of claim 2, further comprising at least one HLA-A primer set selected from a fourth group consisting of:
a primer set having primers of the following sequences: SEQ ID NO: 25, SEQ ID NO: 26, SEQ ID NO: 27, and SEQ ID NO: 28;
a primer set having primers of the following sequences: SEQ ID NO: 16 and SEQ ID NO: 17;
a primer set having primers of the following sequences: SEQ ID NO: 22 and SEQ ID NO: 21;
a primer set having primers of the following sequences: SEQ ID NO: 5, SEQ ID NO: 6, and SEQ ID NO: 7;
a primer set having primers of the following sequences: SEQ ID NO: 10 and SEQ ID NO: 11;
a primer set having primers of the following sequences: SEQ ID NO: 12 and SEQ ID NO: 13; and
a primer set having primers of the following sequences: SEQ ID NO: 1 and SEQ ID NO: 2.

7. The primer array of claim 6 comprising substantially all of the HLA-A primer sets of the fourth group.

8. The primer array of claim 2, further comprising at least one HLA-B primer set selected from a fifth group consisting of:
a primer set having primers of the following sequences: SEQ ID NO: 71 and SEQ ID NO: 72;
a primer set having primers of the following sequences: SEQ ID NO: 58 and SEQ ID NO: 73;
a primer set having primers of the following sequences: SEQ ID NO: 74 and SEQ ID NO: 75;
a primer set having primers of the following sequences: SEQ ID NO: 38 and SEQ ID NO: 39;
a primer set having primers of the following sequences: SEQ ID NO: 40 and SEQ ID NO: 41;
a primer set having primers of the following sequences: SEQ ID NO: 42 and SEQ ID NO: 43;
a primer set having primers of the following sequences: SEQ ID NO: 44 and SEQ ID NO: 45;
a primer set having primers of the following sequences: SEQ ID NO: 38 and SEQ ID NO: 41;
a primer set having primers of the following sequences: SEQ ID NO: 44 and SEQ ID NO: 43;
a primer set having primers of the following sequences: SEQ ID NO: 48 and SEQ ID NO: 49;
a primer set having primers of the following sequences: SEQ ID NO: 50 and SEQ ID NO: 51;
a primer set having primers of the following sequences: SEQ ID NO: 52, SEQ ID NO: 53, and SEQ ID NO: 54;
a primer set having primers of the following sequences: SEQ ID NO: 58 and SEQ ID NO: 49;
a primer set having primers of the following sequences: SEQ ID NO: 32 and SEQ ID NO: 33;
a primer set having primers of the following sequences: SEQ ID NO: 34 and SEQ ID NO: 35;
a primer set having primers of the following sequences: SEQ ID NO: 59, SEQ ID NO: 60, and SEQ ID NO: 61;
a primer set having primers of the following sequences: SEQ ID NO: 62 and SEQ ID NO: 63;
a primer set having primers of the following sequences: SEQ ID NO: 64, SEQ ID NO: 65, SEQ ID NO: 66, and SEQ ID NO: 67;
a primer set having primers of the following sequences: SEQ ID NO: 78 and SEQ ID NO: 79; and
a primer set having primers of the following sequences: SEQ ID NO: 80 and SEQ ID NO: 81.

9. The primer array of claim 8 comprising substantially all of the HLA-B primer sets of the fifth group.

10. The primer array of claim 1 comprising:
a primer set having primers of the following sequences: SEQ ID NO: 23 and SEQ ID NO: 24;
a primer set having primers of the following sequences: SEQ ID NO: 25, SEQ ID NO: 26, SEQ ID NO: 27, and SEQ ID NO: 28;
a primer set having primers of the following sequences: SEQ ID NO: 55, SEQ ID NO: 56, and SEQ ID NO: 57;
a primer set having primers of the following sequences: SEQ ID NO: 68, SEQ ID NO: 69, and SEQ ID NO: 70;
a primer set having primers of the following sequences: SEQ ID NO: 94 and SEQ ID NO: 86; and
a primer set having primers of the following sequences: SEQ ID NO: 95, SEQ ID NO: 96, and SEQ ID NO: 97.

11. A method of processing a DNA sample extracted from a person to analyze personal compatibility, the method comprising:
extracting DNA from the DNA sample;
performing polymerase chain reactions (PCRs) on extracted DNA to amplify human leukocyte antigen (HLA) A, B, and DR genes; and
performing quantitative real-time polymerase chain reactions (qRT-PCRs) on products of the PCRs and on extracted DNA using at least one HLA-A primer set selected to target allele groups of an HLA-A gene, at least one HLA-B primer set selected to target allele groups of an HLA-B gene, and at least one HLA-DRB1 primer set selected to target allele groups of an HLA-DRB1 gene.

12. The method of claim 11, wherein performing the qRT-PCRs comprises:
using two or more HLA-A primer sets selected from a first group consisting of:
a primer set having primers of the following sequences: SEQ ID NO: 23 and SEQ ID NO: 24;
a primer set having primers of the following sequences: SEQ ID NO: 29, SEQ ID NO: 30, and SEQ ID NO: 31;
a primer set having primers of the following sequences: SEQ ID NO: 18, SEQ ID NO: 19, SEQ ID NO: 20, and SEQ ID NO: 21;
a primer set having primers of the following sequences: SEQ ID NO: 8 and SEQ ID NO: 9;
a primer set having primers of the following sequences: SEQ ID NO: 14 and SEQ ID NO: 15; and
a primer set having primers of the following sequences: SEQ ID NO: 3 and SEQ ID NO: 4;
using two or more HLA-B primer sets selected from a second group consisting of:
a primer set having primers of the following sequences: SEQ ID NO: 76 and SEQ ID NO: 77;
a primer set having primers of the following sequences: SEQ ID NO: 46 and SEQ ID NO: 47;
a primer set having primers of the following sequences: SEQ ID NO: 55, SEQ ID NO: 56, and SEQ ID NO: 57;
a primer set having primers of the following sequences: SEQ ID NO: 36 and SEQ ID NO: 37;
a primer set having primers of the following sequences: SEQ ID NO: 68, SEQ ID NO: 69, and SEQ ID NO: 70; and
a primer set having primers of the following sequences: SEQ ID NO: 82, SEQ ID NO: 83, and SEQ ID NO: 84;
using two or more HLA-DRB1 primer sets selected from a third group consisting of:
a primer set having primers of the following sequences: SEQ ID NO: 94 and SEQ ID NO: 86;
a primer set having primers of the following sequences: SEQ ID NO: 95, SEQ ID NO: 96, and SEQ ID NO: 97;
a primer set having primers of the following sequences: SEQ ID NO: 98, SEQ ID NO: 99, SEQ ID NO: 100, and SEQ ID NO: 101;
a primer set having primers of the following sequences: SEQ ID NO: 85 and SEQ ID NO: 86;
a primer set having primers of the following sequences: SEQ ID NO: 89, SEQ ID NO: 90, SEQ ID NO: 91, SEQ ID NO: 92, and SEQ ID NO: 93; and
a primer set having primers of the following sequences: SEQ ID NO: 87 and SEQ ID NO: 88.

13. The method of claim 12, wherein performing the qRT-PCRs comprises using substantially all of the HLA-A primer sets of the first group, substantially all of the HLA-B primer sets of the second group, and substantially all of the HLA-DRB1 primer sets of the third group.

14. The method of claim 12, wherein performing the qRT-PCRs further comprises using at least one HLA-A primer set selected from a fourth group consisting of:
a primer set having primers of the following sequences: SEQ ID NO: 25, SEQ ID NO: 26, SEQ ID NO: 27, and SEQ ID NO: 28;
a primer set having primers of the following sequences: SEQ ID NO: 16 and SEQ ID NO: 17;
a primer set having primers of the following sequences: SEQ ID NO: 22 and SEQ ID NO: 21;
a primer set having primers of the following sequences: SEQ ID NO: 5, SEQ ID NO: 6, and SEQ ID NO: 7;
a primer set having primers of the following sequences: SEQ ID NO: 10 and SEQ ID NO: 11;
a primer set having primers of the following sequences: SEQ ID NO: 12 and SEQ ID NO: 13; and
a primer set having primers of the following sequences: SEQ ID NO: 1 and SEQ ID NO: 2.

15. The method of claim 14, wherein performing the qRT-PCRs comprises using substantially all of the HLA-A primer sets of the fourth group.

16. The method of claim 12, wherein performing the qRT-PCRs further comprises using at least one HLA-B primer set selected from a fifth group consisting of:
a primer set having primers of the following sequences: SEQ ID NO: 71 and SEQ ID NO: 72;
a primer set having primers of the following sequences: SEQ ID NO: 58 and SEQ ID NO: 73;
a primer set having primers of the following sequences: SEQ ID NO: 74 and SEQ ID NO: 75;
a primer set having primers of the following sequences: SEQ ID NO: 38 and SEQ ID NO: 39;
a primer set having primers of the following sequences: SEQ ID NO: 40 and SEQ ID NO: 41;
a primer set having primers of the following sequences: SEQ ID NO: 42 and SEQ ID NO: 43;
a primer set having primers of the following sequences: SEQ ID NO: 44 and SEQ ID NO: 45;
a primer set having primers of the following sequences: SEQ ID NO: 38 and SEQ ID NO: 41;
a primer set having primers of the following sequences: SEQ ID NO: 44 and SEQ ID NO: 43;
a primer set having primers of the following sequences: SEQ ID NO: 48 and SEQ ID NO: 49;
a primer set having primers of the following sequences: SEQ ID NO: 50 and SEQ ID NO: 51;
a primer set having primers of the following sequences: SEQ ID NO: 52, SEQ ID NO: 53, and SEQ ID NO: 54;
a primer set having primers of the following sequences: SEQ ID NO: 58 and SEQ ID NO: 49;
a primer set having primers of the following sequences: SEQ ID NO: 32 and SEQ ID NO: 33;
a primer set having primers of the following sequences: SEQ ID NO: 34 and SEQ ID NO: 35;
a primer set having primers of the following sequences: SEQ ID NO: 59, SEQ ID NO: 60, and SEQ ID NO: 61;
a primer set having primers of the following sequences: SEQ ID NO: 62 and SEQ ID NO: 63;
a primer set having primers of the following sequences: SEQ ID NO: 64, SEQ ID NO: 65, SEQ ID NO: 66, and SEQ ID NO: 67;
a primer set having primers of the following sequences: SEQ ID NO: 78 and SEQ ID NO: 79; and
a primer set having primers of the following sequences: SEQ ID NO: 80 and SEQ ID NO: 81.

17. The method of claim 16, wherein performing the qRT-PCRs comprises using substantially all of the HLA-B primer sets of the fifth group.

18. The method of claim 11, wherein performing the qRT-PCRs comprises using the following primer sets:
a primer set having primers of the following sequences: SEQ ID NO: 23 and SEQ ID NO: 24;
a primer set having primers of the following sequences: SEQ ID NO: 25, SEQ ID NO: 26, SEQ ID NO: 27, and SEQ ID NO: 28;
a primer set having primers of the following sequences: SEQ ID NO: 55, SEQ ID NO: 56, and SEQ ID NO: 57;
a primer set having primers of the following sequences: SEQ ID NO: 68, SEQ ID NO: 69, and SEQ ID NO: 70;
a primer set having primers of the following sequences: SEQ ID NO: 94 and SEQ ID NO: 86; and
a primer set having primers of the following sequences: SEQ ID NO: 95, SEQ ID NO: 96, and SEQ ID NO: 97.


SEQUENCE LISTING (101 sequences)

SEQ ID NO: 1 - DNA, 19 bp, Artificial Sequence
  HLA-A group specific PCR primer HLAA1F
gcgacgccgcgagccagaa

SEQ ID NO: 2 - DNA, 20 bp, Artificial Sequence
  HLA-A group specific PCR primer HLAA1R
gccctccaggtagactctcc

SEQ ID NO: 3 - DNA, 19 bp, Artificial Sequence
  HLA-A group specific PCR primer HLA A G6 F
gcgacgccgcgagccagaa

SEQ ID NO: 4 - DNA, 20 bp, Artificial Sequence
  HLA-A group specific PCR primer HLA A G6 R
ccaggtatctgcggagcccg

SEQ ID NO: 5 - DNA, 20 bp, Artificial Sequence
  HLA-A group specific PCR primer HLAA28/A10F
gtatggctgcgacgtggggt

SEQ ID NO: 6 - DNA, 20 bp, Artificial Sequence
  HLA-A group specific PCR primer HLAA28/A10F2
gtatggctgcgacgtggggc

SEQ ID NO: 7 - DNA, 20 bp, Artificial Sequence
  HLA-A group specific PCR primer HLAA28/A10 -R
ccctccaggtaggctctcca

SEQ ID NO: 8 - DNA, 23 bp, Artificial Sequence
  HLA-A group specific PCR primer HLA A G4 F
ggccggagtattgggaccggaac

SEQ ID NO: 9 - DNA, 21 bp, Artificial Sequence
  HLA-A group specific PCR primer HLA A G4 R
gccctccaggtaggctctcca

SEQ ID NO: 10 - DNA, 25 bp, Artificial Sequence
  HLA-A group specific PCR primer HLA A68 F
gaacacacggaatgtgaaggcccag

SEQ ID NO: 11 - DNA, 22 bp, Artificial Sequence
  HLA-A group specific PCR primer HLA A68 R
gcctcccacttgtgcttggtgg

SEQ ID NO: 12 - DNA, 20 bp, Artificial Sequence
  HLA-A group specific PCR primer HLAA11F
gcttcctccgcgggtaccgg

SEQ ID NO: 13 - DNA, 20 bp, Artificial Sequence
  HLA-A group specific PCR primer HLAA11 -R
ccctccaggtaggctctctg

SEQ ID NO: 14 - DNA, 28 bp, Artificial Sequence
  HLA-A group specific PCR primer HLA A G5 F
ctcccactccatgaggtatttctacacc

SEQ ID NO: 15 - DNA, 22 bp, Artificial Sequence
  HLA-A group specific PCR primer HLA A G5 R
ggccctccaggtaggctctctg

SEQ ID NO: 16 - DNA, 20 bp, Artificial Sequence
  HLA-A group specific PCR primer HLAA3/A19F
gggaccaggagacacggaat

SEQ ID NO: 17 - DNA, 21 bp, Artificial Sequence
  HLA-A group specific PCR primer HLAA3/A19 -R
gccatccaggtaggctctcaa

SEQ ID NO: 18 - DNA, 21 bp, Artificial Sequence
  HLA-A group specific PCR primer HLA A G3 F
ggccggagtattgggaccgga

SEQ ID NO: 19 - DNA, 21 bp, Artificial Sequence
  HLA-A group specific PCR primer HLA A G3 F2
ggccggagtattgggacctgc

SEQ ID NO: 20 - DNA, 21 bp, Artificial Sequence
  HLA-A group specific PCR primer HLA A G3 F3
ggcctgagtattgggaccagg

SEQ ID NO: 21 - DNA, 19 bp, Artificial Sequence
  HLA-A group specific PCR primer HLA A G3/A30/31 R
ggagccactccacgcacgt

SEQ ID NO: 22 - DNA, 24 bp, Artificial Sequence
  HLA-A group specific PCR primer HLA A A30/A31 F
ccgtggatagagcaggagaggcct

SEQ ID NO: 23 - DNA, 19 bp, Artificial Sequence
  HLA-A group specific PCR primer HLAA2F
ggagccccgcttcatcgca

SEQ ID NO: 24 - DNA, 21 bp, Artificial Sequence
  HLA-A group specific PCR primer HLAA2-R
gccctccaggtaggctctcaa

SEQ ID NO: 25 - DNA, 20 bp, Artificial Sequence
  HLA-A group specific PCR primer HLAA9F2
gactgaccgagagagcctgc

SEQ ID NO: 26 - DNA, 20 bp, Artificial Sequence
  HLA-A group specific PCR primer HLAA9F
gactgaccgagagaacctgc

SEQ ID NO: 27 - DNA, 20 bp, Artificial Sequence
  HLA-A group specific PCR primer HLAA9-R2
ccctccaggtaggctctcaa

SEQ ID NO: 28 - DNA, 20 bp, Artificial Sequence
  HLA-A group specific PCR primer HLAA9-R
ccctccaggtaggctctctg

SEQ ID NO: 29 - DNA, 20 bp, Artificial Sequence
  HLA-A group specific PCR primer HLA A G2 F
garcctgcggatcgcgctcc

SEQ ID NO: 30 - DNA, 22 bp, Artificial Sequence
  HLA-A group specific PCR primer HLA A G2 R
gtgccctccaggtaggctctct

SEQ ID NO: 31 - DNA, 22 bp, Artificial Sequence
  HLA-A group specific PCR primer HLA A G2 2R
gtgccctccaggtaggctctca

SEQ ID NO: 32 - DNA, 20 bp, Artificial Sequence
  HLA-B group specific PCR primer HLA B15F
gaccgggagacacagatctc

SEQ ID NO: 33 - DNA, 18 bp, Artificial Sequence
  HLA-B group specific PCR primer HLA B15R
ccttgccgtcgtaggcgg

SEQ ID NO: 34 - DNA, 20 bp, Artificial Sequence
  HLA-B group specific PCR primer HLA B18F
gacgccgcgagtccgaggac

SEQ ID NO: 35 - DNA, 18 bp, Artificial Sequence
  HLA-B group specific PCR primer HLA B18R
ccttgccgtcgtaggcgg

SEQ ID NO: 36 - DNA, 19 bp, Artificial Sequence
  HLA-B group specific PCR primer HLA B G7 F
gagtccgaggayggmgccc

SEQ ID NO: 37 - DNA, 22 bp, Artificial Sequence
  HLA-B group specific PCR primer HLA B G7 R
cccasgtcgcagccrtacatcc

SEQ ID NO: 38 - DNA, 24 bp, Artificial Sequence
  HLA-B group specific PCR primer HLA B37F
gtctcacaccatccagaggatgtc

SEQ ID NO: 39 - DNA, 23 bp, Artificial Sequence
  HLA-B group specific PCR primer HLA B37R
gtaatccttgccgtcgtaggcga

SEQ ID NO: 40 - DNA, 24 bp, Artificial Sequence
  HLA-B group specific PCR primer HLA B8F
gctcccactccatgaggtatttcg

SEQ ID NO: 41 - DNA, 20 bp, Artificial Sequence
  HLA-B group specific PCR primer HLA B8R
ccctccaggtaggctctgtc

SEQ ID NO: 42 - DNA, 20 bp, Artificial Sequence
  HLA-B group specific PCR primer HLA B38F
gaacctgcggatcgcgctcc

SEQ ID NO: 43 - DNA, 20 bp, Artificial Sequence
  HLA-B group specific PCR primer HLA B38R
cacgtgccctccaggtaggt

SEQ ID NO: 44 - DNA, 18 bp, Artificial Sequence
  HLA-B group specific PCR primer HLA B39F
gagcaggaggggccggaa

SEQ ID NO: 45 - DNA, 20 bp, Artificial Sequence
  HLA-B group specific PCR primer HLA B39R
gctctggttgtagtagccgc

SEQ ID NO: 46 - DNA, 20 bp, Artificial Sequence
  HLA-B group specific PCR primer HLA B G2 F
gggcgccgtggatagagcag

SEQ ID NO: 47 - DNA, 19 bp, Artificial Sequence
  HLA-B group specific PCR primer HLA B G2 R
ggagccactccacgcacgt

SEQ ID NO: 48 - DNA, 19 bp, Artificial Sequence
  HLA-B group specific PCR primer HLA B27F
cctgagctcctggaccgcc

SEQ ID NO: 49 - DNA, 20 bp, Artificial Sequence
  HLA-B group specific PCR primer HLA B27R
cgtctccttcccgttctcca

SEQ ID NO: 50 - DNA, 20 bp, Artificial Sequence
  HLA-B group specific PCR primer HLA B40F
cggagtattgggaccgggag

SEQ ID NO: 51 - DNA, 19 bp, Artificial Sequence
  HLA-B group specific PCR primer HLA B40R
ccgcggcggtccaggagcg

SEQ ID NO: 52 - DNA, 21 bp, Artificial Sequence
  HLA-B group specific PCR primer HLA B7F
cacagatctacaaggcccagg

SEQ ID NO: 53 - DNA, 23 bp, Artificial Sequence
  HLA-B group specific PCR primer HLA B7F2
gacacagatctccaagaccaaca

SEQ ID NO: 54 - DNA, 17 bp, Artificial Sequence
  HLA-B group specific PCR primer HLA B7R
cagcgcgctccagcttg

SEQ ID NO: 55 - DNA, 21 bp, Artificial Sequence
  HLA-B group specific PCR primer HLA B G3 F
cgacgccacgagtccgaggaa

SEQ ID NO: 56 - DNA, 20 bp, Artificial Sequence
  HLA-B group specific PCR primer HLA B G3 R
cggagccactccacgcactc

SEQ ID NO: 57 - DNA, 20 bp, Artificial Sequence
  HLA-B group specific PCR primer HLA B G3 F2
gacgccgcgagtccgagaga

SEQ ID NO: 58 - DNA, 19 bp, Artificial Sequence
  HLA-B group specific PCR primer HLA B50F
ggagccccgcttcatcacc

SEQ ID NO: 59 - DNA, 20 bp, Artificial Sequence
  HLA-B group specific PCR primer HLA B44F
ctccgcgggtatgaccagga

SEQ ID NO: 60 - DNA, 20 bp, Artificial Sequence
  HLA-B group specific PCR primer HLA B44F2
ctccgcgggtatgaccagta

SEQ ID NO: 61 - DNA, 20 bp, Artificial Sequence
  HLA-B group specific PCR primer HLA B44R
ctccaggtatctgcggagcg

SEQ ID NO: 62 - DNA, 20 bp, Artificial Sequence
  HLA-B group specific PCR primer HLA B13F
ggctacgtggacgacaccca

SEQ ID NO: 63 - DNA, 19 bp, Artificial Sequence
  HLA-B group specific PCR primer HLA B13R
cgggccgcctcccacttga

SEQ ID NO: 64 - DNA, 20 bp, Artificial Sequence
  HLA-B group specific PCR primer HLA B53F
gacgccgcgagtccgaggac

SEQ ID NO: 65 - DNA, 20 bp, Artificial Sequence
  HLA-B group specific PCR primer HLA B53F2
cgagtccgaggaaggagccg

SEQ ID NO: 66 - DNA, 24 bp, Artificial Sequence
  HLA-B group specific PCR primer HLA B53R
cgcagccatacatcctctggatga

SEQ ID NO: 67 - DNA, 21 bp, Artificial Sequence
  HLA-B group specific PCR primer HLA B53 R2
cagccatacatcstctgccaa

SEQ ID NO: 68 - DNA, 21 bp, Artificial Sequence
  HLA-B group specific PCR primer HLA B G5 F
gcgaggccgggtctcacatca

SEQ ID NO: 69 - DNA, 23 bp, Artificial Sequence
  HLA-B group specific PCR primer HLA B G5 F2
gccgggtctcacacttggcagac

SEQ ID NO: 70 - DNA, 20 bp, Artificial Sequence
  HLA-B group specific PCR primer HLA B G5 R
cggagcsactccacgcacag

SEQ ID NO: 71 - DNA, 20 bp, Artificial Sequence
  HLA-B group specific PCR primer HLA B56F
cgacgccgcgagtccgagag

SEQ ID NO: 72 - DNA, 20 bp, Artificial Sequence
  HLA-B group specific PCR primer HLA B56R
ccgtcgtaggcggactggtc

SEQ ID NO: 73 - DNA, 20 bp, Artificial Sequence
  HLA-B group specific PCR primer HLA B50R
ccgttctccaggtatctgcg

SEQ ID NO: 74 - DNA, 19 bp, Artificial Sequence
  HLA-B group specific PCR primer HLAB G1A F
ggggagccccgcttcatct

SEQ ID NO: 75 - DNA, 20 bp, Artificial Sequence
  HLA-B group specific PCR primer HLAB G1A R
gctgggtgatctgagccgcc

SEQ ID NO: 76 - DNA, 19 bp, Artificial Sequence
  HLA-B group specific PCR primer HLA B G1B F
ggggagccccgcttcatca

SEQ ID NO: 77 - DNA, 20 bp, Artificial Sequence
  HLA-B group specific PCR primer HLA B G1B R
gctgggtgatctgagccgcg

SEQ ID NO: 78 - DNA, 20 bp, Artificial Sequence
  HLA-B group specific PCR primer HLA B57F
ggaacatgaaggcctccgcg

SEQ ID NO: 79 - DNA, 23 bp, Artificial Sequence
  HLA-B group specific PCR primer HLA B57R
ctcgttcagggcgatgtaatcct

SEQ ID NO: 80 - DNA, 23 bp, Artificial Sequence
  HLA-B group specific PCR primer HLA B55F
catgaggtatttctacaccgcca

SEQ ID NO: 81 - DNA, 20 bp, Artificial Sequence
  HLA-B group specific PCR primer HLA B55R
cggtcagtctgtgcctgggc

SEQ ID NO: 82 - DNA, 26 bp, Artificial Sequence
  HLA-B group specific PCR primer HLA B G6 F
ctccatgaggtatttctacaccgcca

SEQ ID NO: 83 - DNA, 19 bp, Artificial Sequence
  HLA-B group specific PCR primer HLA B G6 R
gtctgtgcctggsscttgt

SEQ ID NO: 84 - DNA, 19 bp, Artificial Sequence
  HLA-B group specific PCR primer HLA B G6 R2
gtctgcgcggaggccttca

SEQ ID NO: 85 - DNA, 19 bp, Artificial Sequence
  HLA-D group specific PCR primer DR11F
gctggggcggcctgatgag

SEQ ID NO: 86 - DNA, 22 bp, Artificial Sequence
  HLA-D group specific PCR primer DR11R/2R
cccgtagttgtgtctgcagtag

SEQ ID NO: 87 - DNA, 25 bp, Artificial Sequence
  HLA-D group specific PCR primer DR13F
cacgtttcttggagtactctacgtc

SEQ ID NO: 88 - DNA, 19 bp, Artificial Sequence
  HLA-D group specific PCR primer DR13R
cccgctcgtcttccaggat

SEQ ID NO: 89 - DNA, 25 bp, Artificial Sequence
  HLA-D group specific PCR primer DR14F
cacgtttcttggagtactctacgtc

SEQ ID NO: 90 - DNA, 25 bp, Artificial Sequence
  HLA-D group specific PCR primer DR14F2
cacgtttcttggagtactctacggg

SEQ ID NO: 91 - DNA, 19 bp, Artificial Sequence
  HLA-D group specific PCR primer DR14R
cccgcctctgctccaggag

SEQ ID NO: 92 - DNA, 19 bp, Artificial Sequence
  HLA-D group specific PCR primer DR14R2
cccgcctgtcttccaggag

SEQ ID NO: 93 - DNA, 19 bp, Artificial Sequence
  HLA-D group specific PCR primer DR14R4
cccgcctgtcttccaggaa

SEQ ID NO: 94 - DNA, 19 bp, Artificial Sequence
  HLA-D group specific PCR primer DR2F
cctgtggcagcctaagagg

SEQ ID NO: 95 - DNA, 25 bp, Artificial Sequence
  HLA-D group specific PCR primer DR04/01F
cacgtttcttggagcaggttaaaca

SEQ ID NO: 96 - DNA, 22 bp, Artificial Sequence
  HLA-D group specific PCR primer DR04/01R
cgctgcactgtgaagctctcac

SEQ ID NO: 97 - DNA, 22 bp, Artificial Sequence
  HLA-D group specific PCR primer DR04/01R2
cgctgcactgtgaagctctcca

SEQ ID NO: 98 - DNA, 26 bp, Artificial Sequence
  HLA-D group specific PCR primer DR09F
cacgtttcttgtggcagcttaagttt

SEQ ID NO: 99 - DNA, 26 bp, Artificial Sequence
  HLA-D group specific PCR primer DR09F2
cacgtttcctgtggcagggtaagtat

SEQ ID NO: 100 - DNA, 22 bp, Artificial Sequence
  HLA-D group specific PCR primer DR09R
cccgtagttgtgtctgcagtag

SEQ ID NO: 101 - DNA, 22 bp, Artificial Sequence
  HLA-D group specific PCR primer DR09R2
cccgtagttgtgtctgcacacg